(19) United States
(12) Patent Application Publication
(10) Pub. No.: US 2005/0131403 A1
(43) Pub. Date: Jun. 16, 2005

(54) Polypectomy snare for specimen retrieval

(76) Inventor: Stanley F. Chang, Fresno, CA (US)

Correspondence Address: Richard A. Ryan;RYAN & ENGNATH, Suite 104, 8469 N. Millbrook, Fresno, CA, 93720, US

(21) Appl. No.: 10/735,194

(22) Filed: Dec. 13, 2003

(51) Int. Cl. A61B 18/14

(52) U.S. Cl. 606/47

(57) ABSTRACT

An improved polypectomy snare for removing and retrieving polyps from inside an organ during an endoscopic procedure, such as the colon during a colonoscopy. The improved polypectomy snare has an outer sheath with a tip having a non-planar tip surface shaped and configured so that a cut polyp will better attach to the snare to avoid additional efforts to retrieve the polyp or the loss of the poly. The tip of the outer sheath is configured with one or more spikes having pointed ends to attach to the polyp. In a preferred configuration, the tip has a plurality of spikes extending outwardly from the tip and configured in a generally saw-toothed shape. The spikes can be integral with the outer sheath, directly attached to the outer sheath or part of a cap member that attaches to the sheath.

### BACKGROUND OF THE INVENTION

1. Field of the Invention

The field of the present invention relates to apparatuses and procedures for the removal of polyps and other tissues from inside the gastrointestinal tract during endoscopy procedures. Specifically, the invention described herein relates to an improved snare apparatus used to remove polyps from the colon during colonoscopy. More specifically, the present invention relates to a snare apparatus that is configured to facilitate the retrieval of a polyp which has been separated from the colon.

2. Background

Colonoscopy is an extremely important and prevalent medical procedure, especially in the field of colo-rectal cancer detection. At present, over five million colonoscopies are performed per year in the United States alone. An important aspect of the colonoscopy procedure is in the detection and removal of polyps, which are comprised of soft tissue matter, from the colon. Generally, about two thirds of the polyps found in the colon are adenematous and are considered to be pre-malignant. It is generally believed that the removal of these polyps can prevent cancer formation in the colon. Endoscopic inspection alone is usually not sufficient to determine whether a polyp is adenomatous or of another type, such as hyperplastic, that carries no malignant potential. Instead, medical specialists desire the polyp be sent to a laboratory where it can be microscopically examined to determine whether it is malignant or benign. Generally speaking, therefore, all polyps should be removed from the patient when detected during colonoscopy.

The most commonly used technique for removing polyps involves the insertion of a catheter instrument, primarily biopsy forceps or polypectomy snare, through the biopsy channel of the colonoscope. The polypectomy snare has a control handle operatively connected to one end of a wire that is slidably disposed inside a sheath. The opposite end of the wire has a wire loop portion that is configured to collapse to a closed condition when contracted inside the tip of the sheath by operation of the control handle and expand to an open condition when extended out of the sheath. To remove a polyp, the opened wire loop is placed around the polyp and then gradually closed to, in effect, lasso it. When the wire loop closely surrounds the base of the polyp, an electric current is sent through the wire to cauterize the polyp, which dessicates the tissue to allow the wire to more easily cut through the base of the polyp. Utilizing the control handle, the physician gradually closes the wire loop around the polyp by withdrawing the wire back into the plastic sheath. By the time the polyp is completely cut, the tip of the wire loop is usually completely inside the plastic sheath, with the tip of the cut polyp about one centimeter above the tip of the sheath. Once the polyp is severed from the mucosa of the colon, it needs to be retrieved and placed in a container to be submitted to a laboratory for microscopic examination in order to determine its type and the presence of any malignant or suspicious cells.

Small polyps, such as those that are approximately eight millimeter or less in size, account for the great majority of colonic polyps. These small polyps are small enough to be retrieved by suctioning through the biopsy-suction channel of the colonoscope into a receptacle container, and then into a formalin bottle. The easiest way for retrieval is when the polyp adheres to the tip of the plastic sheath of the snare, in which case it can be simply pulled into the channel and quickly retrieved therefrom. However, in a large minority of cases, the separated polyp does not adhere to the tip of the sheath of the snare, but instead falls inside the colon. While any fallen polyps can usually be found and suctioned through the endoscope, sometimes the searching and the suctioning process can be long and arduous, particularly when a small polyp falls into a difficult to see or reach place. It is not uncommon, however, that the polyp is lost and cannot be retrieved, which is a highly undesirable situation.

The tip of the presently available plastic sheath portion of polypectomy snares is smooth and beveled, with a generally planar surface. The bevel facilitates insertion of the snare through the rubber valve at the top of the biopsy-suction channel. When the directional orientation of the beveled tip is favorable, namely the cut portion of the bevel is facing upward, the bevel can help keep a removed polyp on the tip of the sheath. However, the directional orientation of the sheath is generally a result of happenstance, as the orientation cannot be easily controlled while inside the colon. Not infrequently, the polyp falls off, especially when the bevel faces downward. Various retrieval devices are available to help remove larger polyps that cannot go through the suction channel of the colonoscope. These include net or basket devices, such as the Roth basket, the Nakao snare and others.

Although presently available devices are adapted for the retrieval of polyps from inside the colon, they are generally only effectively used for retrieval of larger polyps. In addition, the presently available devices typically require removal of the poypectomy snare used to cut the polyp from the colonoscope prior to insertion of the retrieval device. Unfortunately, this can make the retrieval of a cut polyp more difficult, as the position of the polyp can become “lost” during the exchange of tools. What is needed, therefore, is an plypectomy snare that is particularly configured for improved retrieval of polyps, particularly small polyps, from inside the colon during colonoscopic procedures so that a greater percentage of polyps can be analyzed by a laboratory to determine if they are benign or malignant. Such an improved polypectomy snare should more easily retrieve polyps cut from the colonic wall to prevent loss of the polyp inside the colon. Ideally, such an polypectomy snare will minimize the amount of labor and time associated with a colonoscopy.

### SUMMARY OF THE INVENTION

The polypectomy snare for specimen retrieval of the present invention provides the benefits and solves the problems identified above. That is to say, the present invention discloses an improved polypectomy snare that is adapted to cause the severed polyp to adhere to the tip of the sheath of the snare without regard to orientation of the snare inside the colon. The improved polypectomy snare is, therefore, one which will cause the severed polyp to predictably adhere to its tip so as to substantially improve the ability of the physician to recover small polyps. To the best of the inventor's knowledge, this type of polyp retrieving device has not been described before. The polypectomy snare of the present invention simplifies retrieval of cut polyps during endoscopic procedures and reduces the labor required for those procedures and, therefore, the likely discomfort of the patient.

In one embodiment of the present invention, the polypectomy snare for specimen retrieval comprises a control handle and longitudinal wire component comprised of an outer sheath and a wire member slidably disposed in the outer sheath. The proximal end of the wire member is operatively connected to the control handle and the distal end of the outer sheath has a tip. The tip has a non-planar tip surface that is shaped and configured to facilitate attachment of the polyp to the polypectomy snare after cutting the polyp from the inside of the organ (i.e., the colon). The tip has one or more spikes extending outwardly from the outer sheath, preferably a plurality of such spikes. The spikes can be made integral with the outer sheath, integral with the tip, attached to a planar tip surface at the distal end of the outer sheath or attached to a cap member that attaches to the outer sheath. In a preferred configuration, the plurality of spikes provide a tip surface that is generally saw-toothed shaped.

Accordingly, the primary objective of the present invention is to provide an improved polypectomy snare for specimen retrieval for use during colonoscopies and other endoscopic procedures that has the features generally described above and more specifically described below in the detailed description.

It is also an important objective of the present invention to provide a polypectomy snare for specimen retrieval that can be utilized to effectively retrieve small polyps removed from inside the colon or other organ.

It is also an important objective of the present invention to provide a polypectomy snare for specimen retrieval that comprises an outer sheath having a modified tip portion to improve the likelihood that a small polyp will adhere or attach to the sheath for removal of the polyps from the colon or other organ.

It is also an important objective of the present invention to provide a polypectomy snare for specimen retrieval that utilizes an outer sheath having a tip portion configured with one or more spikes.

The above and other objectives of the present invention are explained in greater detail by reference to the attached figures and description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a top view of a presently available polypectomy snare showing the wire loop portion extended beyond the tip of the sheath;

FIG. 2 is a top view of an improved polypectomy snare for specimen removal of the present invention showing the wire loop portion retracted inside the modified tip of the sheath;

FIG. 3 is an isolated side view of the tip portion of the improved polypectomy snare of FIG. 2;

FIG. 4 is an isolated side view of an alternative tip portion of the improved polypectomy snare of the present invention;

FIG. 5 is an isolated side view of an alternative tip portion of the improved polypectomy snare of the present invention; and

FIG. 6 is an isolated side view of an alternative tip portion of the improved polypectomy snare of the present invention.

### DETAILED DESCRIPTION OF THE PREFERRED EMBODIMENTS

With reference to the figures where like elements have been given like numerical designations to facilitate the reader's understanding of the present invention, and particularly with reference to the embodiments of the improved polypectomy snare for specimen retrieval of the present invention, identified as numeral 10 in FIG. 2, the preferred embodiments of the present invention are set forth below. The enclosed figures and drawings are merely illustrative of the preferred embodiments and represent several different ways of configuring the present invention. Although specific components, materials, configurations and uses of the present invention are illustrated and set forth in this disclosure, it should be understood that a number of variations to the components and to the configuration of those components described herein and in the accompanying figures can be made without changing the scope and function of the invention set forth herein.

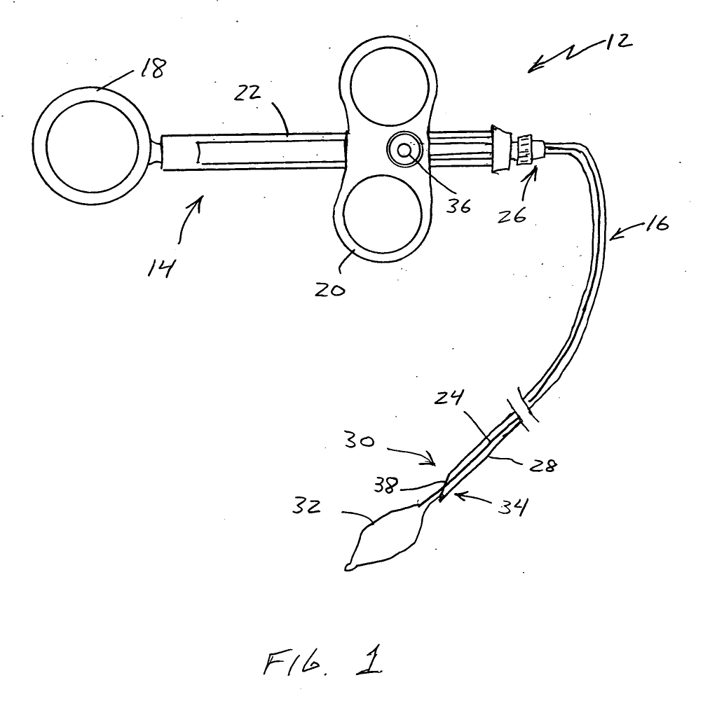

The prior art polypectomy snare, shown as 12 in FIG. 1, comprises a control handle 14 operatively connected to a wire component 16, which is sized and configured to fit within the biopsy channel of the typical endoscope, such as a colonoscope used for colonoscopies. In referring to the opposite ends of the improved polypectomy snare 10 or prior art polypectomy snare 12, the “proximal end” refers to that part of the polypectomy snare 10/12 or wire component 16 that is closest to control handle 14 and the “distal end” refers to that part of the snare 10/12 or wire component 16 farthest from control handle 14. As known to those skilled in the art, the typical control handle 14 comprises a thumb component 18 and a finger component 20 that is slidably disposed on handle body 22. Wire component 16 comprises a wire member 24 operatively connected at the proximal end 26 of wire component 16 to finger component 20 and slidably disposed in an outer sheath 28, typically made out of a plastic material, such that wire member 24 is generally free to move in the direction of the longitudinal axis of sheath 28. The distal end 30 of wire member 24 comprises a collapsible wire loop 32 that is configured to be extended past tip portion 34 of outer sheath 28 and retracted inside sheath 28 by the sliding operation of finger component 20 of handle 12. To remove a polyp, the opened wire loop 32 is placed around the polyp and then wire member 24 is gradually retracted to close wire loop 32 around the polyp. When wire loop 32 closely surrounds the base of the polyp, an electric current is sent through wire member 24, via an electrical connection with electrical connector 36, to cauterize the polyp. The electrical current dessicates the polyp tissue to allow wire member 24 to more easily cut through the base of the polyp. Utilizing control handle 12, the physician gradually closes wire loop 32 around the polyp by withdrawing wire member 24 back into outer sheath 28, thereby cutting the polyp from the organ (i.e., the colon).

As shown in FIG. 1, tip surface 38 of tip 34 of the presently available outer sheath 28 of polypectomy snare 12 is generally configured to be smooth and beveled, with a substantially planar cut across sheath 28. The beveled configuration of tip 34 facilitates insertion of tip 34 through a rubber valve located on the endoscope at or near the top of the endoscope's biopsy-suction channel. As stated above, when the directional orientation of the bevel of tip surface 38 is favorable, namely facing upward, the bevel can help keep a removed polyp on tip 34 of sheath 28. However, as known to those skilled in the art, the directional orientation of sheath 28 is generally a result of happenstance, as the orientation cannot be easily controlled while inside sheath 28 or the colon. Not infrequently, the cut polyp falls off of the smooth, beveled tip surface 38, especially when the bevel portion of tip 34 faces downward. Because of the need for microscopic examination of the polyp to determine whether it is benign or malignant, when the polyp drops off of sheath 28 the physician will usually go to some effort, occasionally significant, to retrieve the polyp.

The polypectomy snare 10 of the present invention is configured to improve the ability of retrieving a cut polyp, shown as 40 in FIG. 2, without the need to resort to additional effort or equipment. As shown in FIG. 2, tip 34 of sheath 28 is shaped and configured to provide a non-smooth, non-planar tip surface 38 that is more beneficial for holding onto a cut polyp 40 that comes in contact therewith. In a preferred embodiment, as shown in FIGS. 2 through 6, tip surface 38 comprises one or more spikes or barb-like members 42 that extend beyond tip 34 of sheath 28. In the preferred embodiment, spikes 42 have a sharp or pointed distal end 44 that is suitable for engaging polyp 40. Tip surface 38 can be configured to comprise a barbed, saw-toothed, spiked or jagged surface (for ease of discussion, these and equivalent non-smooth surfaces are referred to as spiked, having one or more spikes 42). As set forth below, the sharp or pointed end 44 of spikes 42 will stick into polyp 40 and the troughs 46 between ends 44 will help trap and grasp polyp 40. As known to those skilled in the art, spikes 42 can comprise a reverse portion that points generally in the direction of control handle 14 to better engage and hold onto polyp 40. As also known to those skilled in the art, it is preferred that the spikes 42 be relatively short so as to not extend too far beyond tip surface 38 so that they will be less likely to penetrate the colon, even if they are inadvertently directed into the colonic wall.

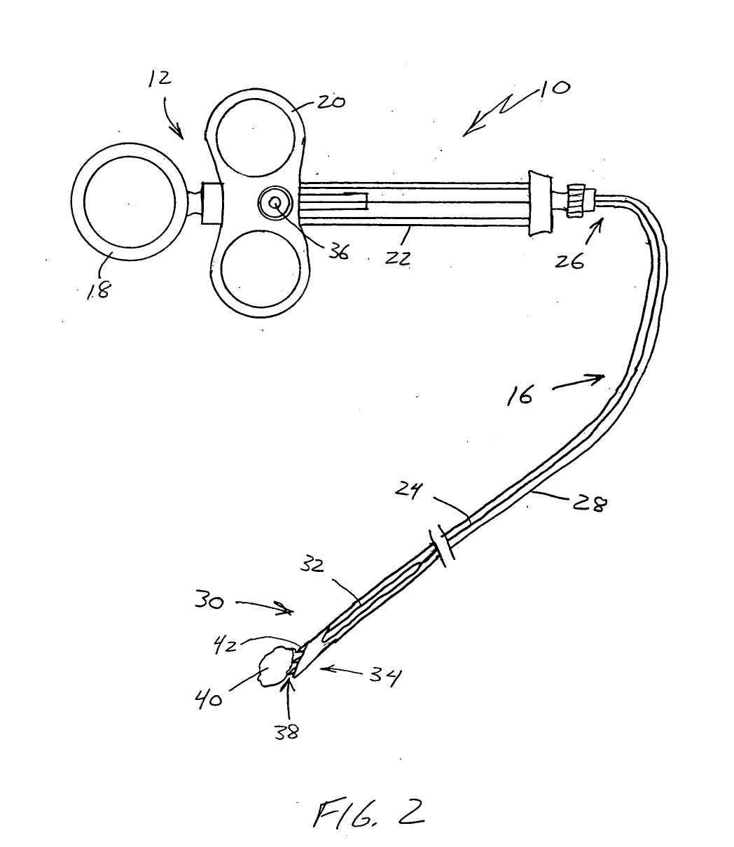

In one configuration, shown in FIGS. 2 and 3, the improved snare 10 comprises a plurality of individual spikes 42 attached to and extending outwardly from tip surface 38 to form a non-planar tip 34 that is more suitable for retrieving polyp 40 than the smooth, generally planar tip surface 38 of the prior art snare 12. Depending on the materials used for outer sheath 28 and spikes 42, attachment of spikes 42 to tip surface 38 can be achieved by adhesives, various bonding techniques or a physical interconnection between spikes 42 and tip surface 38. Those skilled in the art will recognize that other mechanisms for attaching individual spikes 42 to tip surface 38 may also be applicable. Spikes 42 can be made out of the same material as sheath 28, typically a polyethylene or other plastic material, or made out of a non-plastic material. Once attached, spikes 42 form a non-planar tip surface 38 having spike ends 44 suitable for attaching to a cut polyp 40 to avoid loss thereof. It is preferred that spikes 42 be located around the entire circumference of tip surface 38 so that the orientation of wire component 16 in the colon is not important.

In another configuration, shown in FIG. 4, the improved snare 10 comprises a cap member 48 that is configured to be placed over tip 34 of an existing snare 12 and provide a tip surface 38 having one or more spikes 42 thereon. As shown in this figure, cap member 48 can be a molded component that is configured with a first end 50 that can be placed over the smooth tip 34 of the conventional snare 12 (shown with a straight cut edge) and have spikes 42 extending outwardly from the opposite second end 52, creating an improved snare 10 of the present invention. Cap member 48 can be made out of the same material as sheath 28, typically a polyethylene or other plastic material. First end 50 of cap member 48 can have an interior that is slightly larger than the exterior of tip 34 of snare 12 so that it will tightly fit over tip 34. Cap member 48 can be bonded to tip 34 utilizing adhesives or other materials or joining methods suitable for sheath 28 and cap member 48. In a preferred embodiment, the cap member 48 is sized and configured to attach to the tip surface 38 of tip 34 and provide a snare 10 having a smooth transition between sheath 28 and cap member 48 on tip 34. Cap member 48 can be configured to fit over a sheath 28 having a beveled tip 34 or a non-beveled or straight cut tip 34. Whether a straight or bevel cut is provided for cap member 48, it is preferred that spikes 42 be located around the entire circumference of cap member 48 so that the orientation of wire component 16 in the colon is not important.

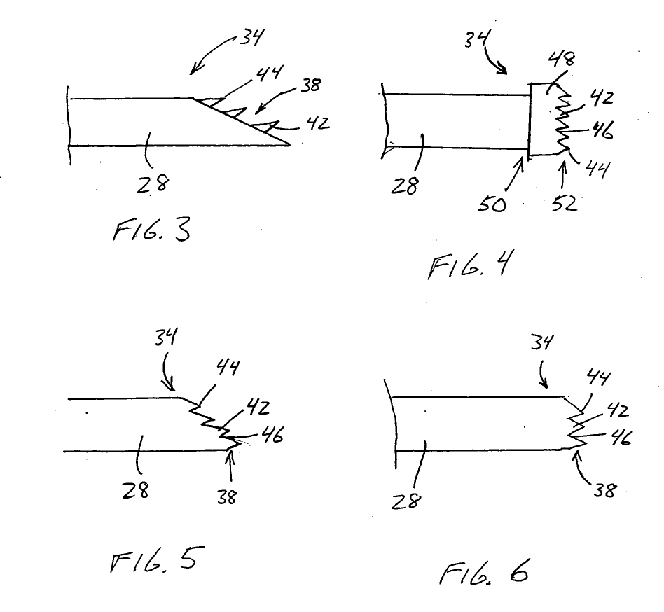

A preferred embodiment, at least for new polypectomy snares, is for spikes 42 of tip 34 to be integral with sheath 28, as shown in FIGS. 5 and 6. This configuration can be achieved by cutting the tip end of sheath 28 with a spiked, saw-toothed, spiked or jagged cut to obtain a plurality of spikes 42 extending outwardly from tip surface 38 or by using a manufacturing process for sheath 28 (i.e., extrusion) that results in a plurality of spikes 42 extending outwardly from tip surface 38 in a generally saw-toothed configuration. An integral configuration avoids the additional cost and manufacturing steps associated with attaching individual spikes 42 (FIG. 3) or cap member 48 (FIG. 4) to the end of sheath 28. As such, this configuration should be somewhat less expensive to manufacture. In addition, an integral tip 34 has the advantage of not providing a potential source of problems, such as those associated with the unintended separation of cap member 48 from sheath 28. Whether a straight or bevel cut is provided for tip 34, it is preferred that spikes 42 be located around the entire circumference of tip surface 38 so that the orientation of wire component 16 in the colon is not important.

As shown in FIGS. 4 and 6 and discussed above, tip 34 of sheath 28 can be a straight edge cut, as opposed to the standard beveled cut shown in FIGS. 2, 3 and 5. In either configuration, polypectomy snare 10 having a plurality of spikes 42 on tip 34 may be more difficult to insert through the rubber inlet valve leading into the biopsy-suction channel of the endoscope. If spikes 42 do make it more difficult, the physician can overcome this problem by pushing the distal end of wire loop 32 slightly out of tip 34 beyond spike ends 44 to serve as the leading end of wire component 16. This can be accomplished by slightly moving the finger component 20 of handle 14 slightly forward. To facilitate this operation, handle 14 can be modified to include a mark thereon for valve insertion and/or be made to “click” at the proper insertion point.

In use, wire component 16 of improved polypectomy snare 10 is inserted into the endoscope inlet valve for the biopsy-suction channel and directed to the distal end of the endoscope and the polyp 40 in the same manner as the presently available polypectomy snare 12. Upon approaching polyp 40, wire loop 32 is pushed out of sheath 28 to lasso the polyp 40. A portion of tip 34 will be in contact with the base of polyp 40 as the wire loop 32 is retracted against the opposite side of polyp 40. Because spikes 42 are around the circumference of tip surface 38, a least some portion of the spikes 42 will be engaged with the polyp when it is cut from the colonic wall. Because spike ends 44 are sharp or pointed, the soft tissue of polyp 40 will attach to spikes 42, allowing much improved retention of polyp 40 for retrieval from inside the colon. Reducing or eliminating the need to search for and separately retrieve a polyp will reduce the time, effort and discomfort associated with a colonoscopy. While the polypectomy snare 10 of the present invention is most applicable to colonoscopy, it is also applicable to other types of endoscopy procedures, such as gastroscopy and the like.

While there are shown and described herein certain specific alternative forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to the dimensional relationships set forth herein and modifications in assembly, materials, size, shape and use.

1. An improved polypectomy snare for removing and retrieving a polyp, said polypectomy snare comprising:
a control handle;
a wire component having a proximal end and a distal end, said proximal end operatively connected to said control handle, said wire component having a wire member slidably disposed in an outer sheath, said outer sheath having a tip at said distal end of said wire component, said tip having a non-planar tip surface, said non-planar tip surface shaped and configured to facilitate attachment of said polyp to said polypectomy snare.

2. The polypectomy snare of claim 1, wherein said tip has one or more spikes extending outwardly from said outer sheath.

3. The polypectomy snare of claim 2, wherein said spikes are integral with said outer sheath.

4. The polypectomy snare of claim 2, wherein said spikes are integral with said tip.

5. The polypectomy snare of claim 2, wherein said spikes are attached to a planar tip surface at the distal end of said outer sheath.

6. The polypectomy snare of claim 1, wherein said tip surface is saw-toothed shaped.

7. The polypectomy snare of claim 1 further comprising a cap member having a first end attached to said outer sheath and a second end having one or more spikes extending outwardly from said cap member.

8. An improved polypectomy snare for retrieving a polyp, said polypectomy snare comprising an outer sheath with a proximal end and a distal end, said outer sheath having a tip at said distal end, said tip configured with a non-planar tip surface, said non-planar tip surface shaped and configured to facilitate attachment of said polyp to said outer sheath.

9. The polypectomy snare of claim 8, wherein said tip has one or more spikes extending outwardly from said outer sheath.

10. The polypectomy snare of claim 9, wherein tip has a plurality of spikes extending outwardly from said outer sheath in a generally saw-toothed configuration.

11. An improved polypectomy snare for retrieving a polyp, said polypectomy snare comprising an outer sheath with a proximal end and a distal end, said outer sheath having a tip, said tip configured with one or more spikes extending outwardly from said tip, said one or more spikes forming a non-planar tip surface, said non-planar tip surface shaped and configured to facilitate attachment of said polyp to said outer sheath.
